(19) United States
(12) Patent Application Publication
(10) Pub. No.: US 2024/0409502 A1
(43) Pub. Date: Dec. 12, 2024

(54) METHODS FOR PRODUCING (POLY)DIAMINODIPHENYLMETHANE AND (POLY)DIPHENYLMETHANE DIISOCYANATE

(71) Applicant: PUBLIC JOINT STOCK COMPANY "SIBUR HOLDING" (PJSC "SIBUR HOLDING"), Tobolsk (RU)

(72) Inventors: Denis Alekseevich LENEV, Tomsk (RU); Valeriya Aleksandrovna MAS, Rostovskaya (RU); Roman Nikolaevich NAUMOV, Tomsk (RU); Anastasiya Aleksandrovna BOBRIKOVA, Moscow (RU); Alexey Andreevich IOFFE, Moscow (RU)

(21) Appl. No.: 18/697,551

(22) PCT Filed: Oct. 12, 2022
(86) PCT No.: PCT/RU2022/050323

§ 371 Date: Apr. 1, 2024

(30) Foreign Application Priority Data
Oct. 13, 2021 (WO) PCT/RU2021/000435

(51) Int. Cl. C07C 263/10; C07C 209/78; C07C 209/86; C07C 211/50; C07C 265/14

(52) U.S. Cl. CPC C07C 263/10; C07C 209/78; C07C 209/86; C07C 211/50; C07C 265/14

(57) ABSTRACT

The invention relates to the field of producing isocyanates, in particular, to a method for producing (poly)diaminodiphenylmethane used to obtain polyisocyanates. The invention relates to a method for changing a molecular weight distribution of pDADPM by recycling a part of dimeric components to the synthesis step. The invention also relates to a method for producing (poly)diphenylmethane diisocyanate by reacting the obtained (poly)diaminodiphenylmethane with phosgene, and to the (poly)diphenylmethane diisocyanate obtained by this method.

### TECHNICAL FIELD

The invention relates to the field of (poly)diaminodiphenylmethane production. In particular, the invention relates to a method for producing (poly)diaminodiphenylmethane having an increased amount of trimeric diaminodiphenylmethane oligomers. The invention also relates to a method for producing (poly)diphenylmethane diisocyanate having an increased amount of trimeric components by reacting (poly)diaminodiphenylmethane with phosgene.

### BACKGROUND

Polyurethanes are used in many fields of industry. This is possible due to the fact that their mechanical properties can be varied by using structurally different isocyanates as a raw material.

One of the most common isocyanates is diphenylmethane diisocyanate (methylene diphenyldiisocyanate, MDI). A starting component for the production of MDI is diaminodiphenylmethane prepared by condensation of aniline and formaldehyde in the presence of a catalyst. The condensation reaction of aniline and formaldehyde gives a mixture of diaminodiphenylmethanes, including (poly)diaminodiphenylmethane (hereinafter also referred to either as pDADPM or as pMDA).

Phosgenation of pMDA results in (poly)diphenylmethane diisocyanate (pMDI). The MDI so obtained is subjected to distillation to isolate pure dimeric MDI and to obtain the final pMDI with a desired molecular weight distribution. This results in separation of a mixture of dimeric MDI and a bottom product, pMDI.

However, the demand for pMDI, in contrast to dimeric MDI, is growing. Therefore, there is a need to develop methods for producing pMDI of a desired functionality and molecular weight without producing a second product, monomeric MDI.

A method for the synthesis of polyamines and polyisocyanates of a desired viscosity by adjusting the aniline to formaldehyde ratio is known, for example, as described in U.S. Pat. No. 4,792,624A patent (publ. 20.12.1988, DOW CHEMICAL CO [US]). A disadvantage of the method is a high content of heavy oligomers (the tetramer-to-trimer ratio is more than 0.50).

Along with this, the corresponding isocyanates may have a reduced reactivity. Thus, the document [The polyurethanes book, David Randall, Steve Lee, Eds., Wiley, 2002, Huntsman International LLC, Polyurethanes business] teaches that the reactivity of internal isocyanate groups is 0.15 to 0.2 in comparison with terminal ones.

The proportion of terminal groups, for example, in an MDI trimer is ⅔ (67%), while in an MDI pentamer this proportion is ⅖ (40%) only. In addition, heavy oligomers of pMDI may be more prone to the formation of resins and, at an increased concentration, can lead to a decrease in the shelf life of a pMDI product.

JP 4292560 B2 (publ. 08.07.2009, NIPPON POLYURETHANE IND CO LTD [JP]) discloses a method for producing polyisocyanates enriched with tri- and tetrafunctional oligomers by extraction with supercritical CO₂. A disadvantage of the method is the need to utilize a byproduct stream containing heavy oligomers and resins insoluble in CO₂.

In addition, the trimer/dimer ratio in polyamine can be partially changed by stripping 2,2′- and 2,4′-dimers from polyamine (at least 80% of the sum of 2,2′- and 2,4′-dimers) and recycling them to the step of synthesis as disclosed, for example, in EP 1167343 A1 (publ. 02.01.2002, BASF AG [DE]). However, this method is not too effective in influencing the molecular weight distribution due to a low content of 2,2′- and 2,4′-dimers in a polyamine mixture (no more than 3-5%). Another disadvantage of the method is the need to use columns with a large number of separation steps (50 steps) to isolate a mixture of dimers, which is essentially free of 4,4′-dimer.

A method for recycling dimers into a reaction with a reduced number of separation steps is known (WO 2017125302 A1, publ. 27.07.2017, BASF SE [DE]). This method is characterized by the separation of a stream of impurities from the recycled dimers. The impurities can include aminobenzylamines, mono-methyl-MDA, formanilide, and dihydroacridines. A disadvantage of the method is the need for expensive disposal of the stream comprising impurities. Another disadvantage of the method is a relatively high number of separation steps (10 to 20) required to separate a purified mixture to be recycled containing not more than 20% of 4,4′-dimer.

A method for producing at least 98% by weight of pure 4,4′-diisocyanatodiphenylmethane from the phosgenation product of a mixture of polyamines obtained by condensation of aniline and formaldehyde is known (GB1263439 A, publ. 09.02.1972, BAYER AG [DE]). Pure 4,4′-diisocyanatodiphenylmethane is obtained by fractional separation of isocyanates. A disadvantage of the method is the impossibility of varying the isocyanate composition at the step of preparing polyamines.

Distillation of 4,4′-dimer of polyamine and utilization thereof in another production process, for example, in the production of epoxy resins could be another variant of changing the molecular weight distribution of polyisocyanate at the polyamine step. However, this method requires the integration of two production processes and is not always possible.

Thus, the development of a component-balanced method for producing pMDA and pMDI with an improved oligomeric distribution remains an urgent task.

### SUMMARY OF THE INVENTION

An objective of the present invention is to develop a method for producing (poly)diaminodiphenylmethane having a reduced amount of heavy oligomers and an increased amount of trimer, and to obtain (poly)diphenylmethane diisocyanate (pMDI) based on a new polyamine.

A technical result resides in changing the molecular weight distribution of polyisocyanate at the polyamine step, namely, reducing the content of heavy oligomers in the polyamine (the tetramer-to-trimer weight ratio is less than 0.48, while the weight ratio of trimer to the sum of dimers is 0.45 or more), while maintaining a viscosity of polyamine of from 50 to 150 mPa·s at 90° C. and a viscosity of the resulting polyisocyanate of from 150 to 250 mPa·s at 25° C., and also improving the quality of (poly)diphenylmethane diisocyanate.

The technical result also resides in reducing the amount of aniline in a phosgenated polyamine to the level of less than 10-20 ppm by weight, which leads to a decrease in the content of phenyl isocyanate in pMDT isocyanate.

An additional technical result resides in elimination of the need to build a unit for the separation of dimeric methylene phenyldiisocyanate from pMDI.

The above technical problem is solved and the technical result is achieved by performing the condensation of aniline and formaldehyde in the presence of HCl as a catalyst, with introduction of a mixture of recycled isomeric diaminodiphenylmethanes (hereinafter also referred to either as DADPM or as MDA) into the reaction mass. At that, in one embodiment of the invention, the molar ratio of chlorine contained in HCl to the total nitrogen contained in aniline and recycled MDAs, Cl/N=(0.2÷0.4)/1.

Without being bound by any theory, the inventors believe that polyamine (pMDA) is formed by polycondensation of formaldehyde (CH₂O) with aniline, with the release of water and the formation of a mixture of primary amines having the structure of poly-(methylene-2,4-phenylene-1(5)-amine) (Scheme 1):

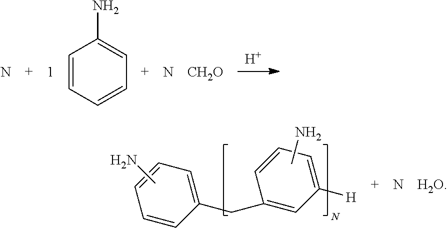

Scheme 1—Polycondensation of Aniline and Formaldehyde to Obtain pMDA

The molecular weight distribution of polycondensation products, including pMDA, is determined by the Flory-Schulz distribution (equation 1):

$$P ( k ) = {{ka}^{2} ( 1 - a )}^{k - 1} , \qquad ( 1 )$$

where P(k) is a molar fraction of a component with the polymerization degree k, a is an empirical parameter. The molecular weight distribution of polyamine, starting from dimeric diamine (M2A, and further MkA), for different a is shown in Table 1, based on the weight fractions of the corresponding polyamine components.

TABLE 1
Flory-Schulz weight distribution of polyamine for different a

| | | | | | | |
|---|---|---|---|---|---|---|
| A | 0.74 | 0.75 | 0.76 | 0.77 | 0.80 | 0.82 |
| M2A | 49.00 | 50.63 | 52.29 | 53.98 | 59.21 | 62.84 |
| M3A | 28.99 | 28.81 | 28.56 | 28.25 | 26.95 | 25.74 |
| M4A | 13.48 | 12.88 | 12.26 | 11.62 | 9.64 | 8.29 |
| M5A | 5.49 | 5.05 | 4.61 | 4.19 | 3.02 | 2.34 |
| M6A | 2.06 | 1.82 | 1.60 | 1.39 | 0.87 | 0.607 |
| M7A | 0.73 | 0.62 | 0.52 | 0.44 | 0.24 | 0.15 |
| M4/M3 | 0.465 | 0.447 | 0.429 | 0.411 | 0.358 | 0.322 |
| M3/M2 | 0.592 | 0.569 | 0.546 | 0.523 | 0.455 | 0.410 |

On the other hand, to obtain pMDI with a suitable viscosity, for example, in a range between 150 and 250 mPa·s at 25° C., the content of dimeric components must be not more than 52% (U.S. Pat. No. 4,792,624 A, publ. 20.12.1988, DOW CHEMICAL CO [US]). At a theoretical molecular weight distribution, according to Table 1, with a decrease in the fraction of the dimeric component in polyamine to less than 52%, the theoretically and practically achievable tetramer/trimer ratio will be at least 0.465 (a=0.74) and a trimer/dimer ratio of more than 0.592. However, these values of higher yield of trimer, low enough for tetramer/trimer ratio, and high enough for trimer/dimer ratio, are not easily obtained in practice. Thus, in U.S. Pat. No. 4,792,624 A, usual weight ratios of tetramer to trimer are above 0.50, and trimer to dimer of MDA weight ratios are below 0.5. This brings usual content of trimer in polyMDA to 23-24% or below.

Therefore, the reaction is often carried out at low formaldehyde-to-aniline ratios to obtain lighter polyamines, for example, with a=0.80 or 0.82, which are further phosgenated, and a part of the dimeric component is distilled off already at the step of isocyanate synthesis [PERP 2016-3—Nitrobenzene/Aniline/MDI, Nexant Report].

### DETAILED DESCRIPTION

Various aspects of the present invention are disclosed herein below.

Thus, in one aspect, the present invention is directed to (poly)diaminodiphenylmethane having an oligomeric distribution characterized by a tetramer/trimer ratio of not more than 0.48 and a trimer/dimer ratio of not less than 0.45, and a viscosity of from 50 to 150 mPa·s at 90° C. In particular, said polyamine may have a tetramer/trimer weight ratio of not more than 0.42 and/or a trimer/dimer weight ratio of not less than 0.53.

In another aspect, the present invention is directed to a method for producing (poly)diaminodiphenylmethane, comprising the following steps (variant 1):
1) mixing HCl and aniline and a mixture of recycled dimeric diaminodiphenylmethane (DADPM) isomers;
2) reacting the mixture of said aniline, dimeric diaminodiphenylmethane (DADPM) isomers and HCl with formaldehyde at a temperature of not higher than 70° C.;
3) raising the temperature of the reaction mass obtained in step (2) to a temperature below or equal to 140° C. and keeping the reaction mass at this temperature;
4) neutralizing acidic compounds in the mass obtained in step (3);
5) washing the mass obtained in step (4) with water;
6) distilling off low-boiling components and aniline from the mass obtained in step (5); and
7) distilling off the mixture of dimeric diaminodiphenylmethane (DADPM) isomers from the mass obtained in step (6),
characterized in that a stream of dimeric diaminodiphenylmethane (DADPM) isomers obtained in step (7) is recycled to step (1).

In another aspect, the invention further relates to a method for producing (poly)diaminodiphenylmethane, the method comprising the following steps (variant 2):
1) mixing HCl and aniline with addition of a part of a recycled mixture of dimeric diaminodiphenylmethane (DADPM) isomers of from 0 to 25 wt. % based on the total amount of the added recycled mixture;
2) reacting the mixture from step (1) with a first part of an aqueous formaldehyde solution at a temperature T1 of not higher than 70° C., wherein said first part of an aqueous formaldehyde solution is from 50 to 100% of the total amount of formaldehyde added to the reaction;
3) adding the remaining part of the recycled mixture of dimeric diaminodiphenylmethane isomers up to reaching the total amount of DADPM isomers of not more than 25 wt % of the initial weight of aniline;
4*) in the case when in step (2) the introduced amount of formaldehyde is less than 100% of the total amount of formaldehyde introduced into the reaction, adding a second part of formaldehyde of from more than 0 to up to 50% of the total amount of formaldehyde introduced into the reaction;
4) keeping the reaction mass obtained in step 3 or, in the case when step (4*) is carried out, the reaction mass obtained in step (4*) at a temperature T2 of not higher than 80° C.;
5) raising the temperature of the reaction mass obtained in step (4) to a temperature below or equal to 140° C. and keeping it at this temperature;
6) neutralizing acidic compounds in the mass obtained in step (5);
7) washing the mass obtained in step (6) with water;
8) distilling off low-boiling components and aniline from the mass obtained in step (7); and
9) distilling off the mixture of dimeric diaminodiphenylmethane (DADPM) isomers from the mass obtained in step (8),
characterized in that the mixture of dimeric diaminodiphenylmethane (DADPM) isomers obtained in step (9) is recycled to step 1 and/or step 3.

In another aspect, the invention relates to (poly)diaminodiphenylmethane obtained by any of the above methods. The polyamine so produced is characterized by an improved oligomeric distribution, in particular, a tetramer/trimer weight ratio of not more than 0.48 and a trimer/dimer weight ratio of not less than 0.45, and by a viscosity of 50 to 150 mPa·s at 90° C. In particular, said polyamine may have a tetramer/trimer weight ratio of not more than 0.42 and/or a trimer/dimer weight ratio of not less than 0.53.

In still other aspect, the present invention relates to a method for producing (poly)diphenylmethane diisocyanate, comprising the following steps:
a) obtaining (poly)diaminodiphenylmethane according to any of the above methods;
b) phosgenating the (poly)diaminodiphenylmethane obtained in step a) to produce (poly)diphenylmethane diisocyanate.

The main aspects of the implementation of the method for producing (poly)diaminodiphenylmethane according to variant 1 are disclosed below in more detail.

### Step (1): Mixing Aniline, DADPM Isomers and HCl (Preparation of a Solution of Partially Neutralized Amine Hydrochlorides)

Aniline fed to step 1) is pure or recycled aniline, which, inter alia, may contain up to 7% water.

Recycled dimeric DADPM isomers fed to step 1) can be either a solid, a concentrated solution in aniline, or a melt. DADPM isomers may contain aniline and 4,4′-, 2,2′- and 2,4′-isomers in various proportions, as well as impurities and homologues. Preferably, a mixture of dimeric diaminodiphenylmethanes with a content of 4,4′-DADPM of not less than 50% is used as DADPM isomer.

The ratio of aniline to recycled DADPM isomers can be any convenient ratio; however, for economic reasons, it usually does not exceed a weight ratio of 50% DADPM isomers to 50% aniline.

HCl is used in the form of an aqueous solution, i.e. hydrochloric acid, or in the form of hydrogen chloride gas. Hydrochloric acid at a concentration of 20 to 40% can be added alone or mixed with aniline and/or DADPM isomers in any suitable reaction vessel. Hydrogen chloride gas can be used as dry or wet gas. Preferably, hydrochloric acid is used, with a concentration of 31-38% being preferred. In a preferred embodiment of the invention, hydrochloric acid or hydrogen chloride gas is used in such an amount that the molar ratio of Cl/N is from 0.2 to 0.4.

It is preferred that the resulting mixture of water, amine hydrochlorides and neutral amines is a homogeneous solution. Although less preferably, the resulting mixture also can be used in the form of a suspension of hydrochlorides.

Step (2): Reacting the Mixture of Aniline, DADPM Isomers, and HCl with Formaldehyde.

Formaldehyde is used in the form of an aqueous or water-methanol solution, in gaseous form, or in the form of solid paraformaldehyde, preferably in the form of a 30-50% water-methanol solution (usually referred to as formalin).

According to one embodiment of the invention, formaldehyde is used in an amount of 0.4 to 0.7 mol per mol of starting aniline, preferably 0.45 to 0.55 mol per mol of initial aniline.

The reaction of aniline, DADPM isomers, and formaldehyde in the presence of HCl is carried out by any method and under conditions known in the art. Examples of such methods include, but are not limited to, the methods described in: U.S. Pat. No. 5,053,539A (publ. 01.10.1991, MITSUI TOATSU CHEMICALS [JP]), U.S. Pat. No. 9,701,617B2 (publ. 11.07.2017, COVESTRO DEUTSCHLAND AG [DE]). The reaction is carried out at a temperature not higher than 70° C., preferably at a temperature of from 20 to 70° C., for example from 30 or 40° C. to 60° C.

The reaction of aniline, DADPM isomers, HCl, and formaldehyde is carried out in any apparatus known in the art. For example, such a reaction can be carried out in a stirred reactor.

### Step (3): Raising the Temperature and Keeping the Reaction Mass Obtained in Step (2).

The reaction mass obtained in step (2) is subjected to keeping at elevated temperatures. The temperature is preferably raised smoothly. “Smoothly” hereinafter means a rise in temperature at a rate of not more than 5° C. per minute, preferably not more than 1° C. per minute. The keeping is carried out at a temperature range below or equal to 140° C., preferably in a temperature ranging from 80 to 140° C., for example from 90 to 130° C., to complete the rearrangement reaction of the starting reaction mass comprising aminals and benzylamines to a mixture of primary amines. Preferably, the rearrangement should proceed to a benzylamine conversion of 99.9% or more, as determined by NMR spectroscopy. The keeping can be carried out in a stirred tank reactor, a tank vessel, or a tubular displacement reactor.

### Step (4): Neutralizing Acidic Compounds.

The step of neutralizing hydrochloric acid is generally carried out at a temperature of from 60 to 140° C., preferably from 90 to 130° C. Carrying out neutralization at temperatures below 60° C. generally promotes an increase in the viscosity, while carrying out neutralization at temperatures above 140° C. can lead to undesirable dissolution of the organic phase in the aqueous phase. In one embodiment, the neutralization step (4) may be carried out in the presence of aniline as additional diluent. When using additional aniline, its amount is preferably between more than 0 and up to 50%, more preferably between more than 0 and 30%, based on the amount of (poly)diaminodiphenylmethane.

The neutralization step can be carried out in any apparatus known in the art, which allows efficient mixing and subsequent phase separation. Thus, according to one embodiment of the invention, neutralization is carried out in a separator or dynamic extractor with simultaneously supplying acidic (poly)diaminodiphenylmethane, a neutralizing agent and, optionally, aniline or a preliminary prepared mixture of aniline and acidic (poly)diaminodiphenylmethane. According to another embodiment of the invention, the streams of acidic (poly)diaminodiphenylmethane and a neutralizing agent are mixed in a mixer, transferred to a tank vessel (settler) and then are directed to separation.

In one embodiment of the invention, the neutralization is carried out portion-wise, wherein the main portion of alkali (more than 100% of the HCl molar equivalent) is introduced during the first part of the neutralization.

A part of the alkali can be introduced in subsequent steps for a more complete neutralization of formic acid, which is chemically bound in (poly)diaminodiphenylmethane in the form of formamides [Henri Ulrich, Chemistry and Technology of isocyanates, John Wiley and Sons, Chichester, 1996].

Step (5): Washing the Mass Obtained in Step 4 with Water.

After neutralization, the (poly)diaminodiphenylmethane is washed with water.

In the context of the present invention, the water used for washing can be, but is not limited to, distilled, deionized, demineralized, osmotic, bidistilled water or other fresh water purified from iron ions and other ionic and molecular impurities. The amount of water can be from 10 to 500 wt %, preferably from 20 to 200 wt %, based on the mixture of the neutralized mass and aniline.

The washing temperature can be 60 to 100° C. The preferred washing temperature should be high enough to avoid emulsion formation and low enough to reduce the solubility of polyamine components.

### Step (6): Distilling Off Low-Boiling Components and Aniline

The distillation of low-boiling components, which include, but are not limited to, methanol, water, and aniline-water azeotrope, can be carried out in any suitable equipment, for example, in a rectification column, separator, at a suitable temperature and pressure, while providing sufficient heat for evaporation. For example, the distillation temperature can be from 90 to 180° C., and the pressure can be in the range of from atmospheric pressure to 50 mbar.

Distillation of aniline is carried out after distillation of low-boiling components. For the distillation of aniline, the temperature of the bottom of the column is usually raised to the temperature of from 180 to 250° C. at a pressure in the head of the column of from 1 to 50 mbar.

### Step (7): Distilling Off a Mixture of Dimers—Diaminodiphenylmethanes

This step can be carried out in any suitable equipment, for example, a rectification column, usually at a bottom temperature of the column of from 180 to 250° C. and a pressure in the head of the column of from 1 to 50 mbar.

The main aspects of the implementation of the method for producing (poly)diaminodiphenylmethane according to variant 2 are disclosed below in more detail.

Step (1) of variant 2 is similar to step (1) of variant 1, except that recycled isomeric diaminodiphenylmethanes are not added or added in an amount of from more than 0 to 25 wt % based on the total amount of the added recycled mixture.

Step (2) of variant 2 is similar to step (2) of variant 1, except that formaldehyde can be introduced in the whole amount, i.e. in an amount of 100% of the total amount of formaldehyde introduced into the reaction, or it can be introduced as a first portion in an amount of from 50 to less than 100% of the total amount of formaldehyde introduced into the reaction.

Step (3): Adding the remaining part of the recycled mixture of isomeric diaminodiphenylmethanes (in the case when DADPM isomers are not added or added partly in the first step).

The remaining part of the recycled mixture of isomeric diaminodiphenylmethanes (DADPM or MDA) being from 75 to 100 wt. % is added to the reaction mass obtained in step (2) such that the total amount of DADPM isomers added to the mass reaches a value of not more than 25 wt. % of the initial weight of aniline, for example, reaches a value of from 5 wt. %, or from 10 wt. %, or from 15 wt. % to 25 wt. %, or to 20 wt. %, based on the starting weight of aniline.

The recycled DADPM isomers fed to step (1) can be supplied either as a solid, as a concentrated solution in aniline, or as a melt. DADPM isomers may comprise aniline and 4,4′-, 2,2′- and 2,4′-isomers in various proportions, as well as impurities and homologues. Preferably, a mixture of dimeric diaminodiphenylmethanes with a content of 4,4′-DADPM of not less than 50% is used as DADPM isomer.

### Step (4*): Adding a Second Part of the Formaldehyde Aqueous Solution.

In the case when a part of formaldehyde is used in step 2, i.e. less than 100% formaldehyde, a second part of formaldehyde is added after step (3). The second part of formaldehyde is from more than 0% to up to 50% of the total amount of formaldehyde introduced into the reaction.

The addition is usually carried out at a temperature of from 30 to 80° C., preferably from 40 to 80° C.

### Step (4): Raising the Temperature and Keeping the Reaction Mass Obtained in Step 3 or (4*).

The reaction mass obtained in step 3 or, in the case when step (4*) is carried out, the reaction mass obtained in step 4* is subjected to keeping at elevated temperatures. The temperature is preferably raised smoothly. “Smoothly” hereinafter means a rise in temperature at a rate of not more than 5° C. per minute, preferably not more than 1° C. per minute. The keeping is usually performed at a temperature of not higher than 80° C., for example in a temperature ranging from 30 to 80° C., preferably from 40 to 70° C.

### Steps (5)-(9) of Variant 2 are Similar to Steps (3)-(7) of Variant 1.

(Poly)diaminodiphenylmethane obtained according to the above steps is used to prepare (poly)diphenylmethane diisocyanate.

The present invention also relates to a method for producing (poly)diphenylmethane diisocyanate, comprising reacting (poly)diaminodiphenylmethane obtained according to the present invention with phosgene to obtain (poly)diphenylmethane diisocyanate; and to (poly)diphenylmethane diisocyanate obtained by the described method.

(Poly)diphenylmethane diisocyanate is prepared by the interaction between (poly)diaminodiphenylmethane and phosgene according to a phosgenation reaction.

According to one embodiment of the invention, phosgene is used in gaseous form. According to another embodiment of the invention, phosgene is used in dissolved form, where the solvent is a solvent known in the prior art, specific examples of which will be described below.

According to another embodiment of the invention, phosgenation is carried out in gas phase as described, for example, in documents EP1509496A1 (publ. 02.03.2003 BASF AG [DE]), RU2487115C2 (publ. 10.07.2013, BAYER [DE]), and RU2361856C2 (publ. 20.07.2009 BAYER [DE]).

According to another embodiment of the invention, phosgenation is carried out in the presence of an inert solvent, as described, for example, in: U.S. Pat. No. 5,925,783A (publ. 07.20.1999, BAYER [DE]), WO2010149544A2 (publ. 12.29.2010, BASF SE [DE]). Hereinafter, “inert solvent” means a solvent that does not react with the starting and intermediate compounds, as well as with the reaction products. Examples of such solvents include, but are not limited to, chlorobenzene, dichlorobenzene, trichlorobenzene, toluene, dioxane, dimethyl sulfoxide, xylenes, chloroethylbenzene, monochlorobiphenyl, naphthyl chloride, dialkyl phthalates, or mixtures thereof. Chlorobenzene and dichlorobenzene are preferable solvents. The concentration of polyamine in a solvent, if used, is generally from 10 to 40 wt %, preferably from 12 to 25 wt %.

In another embodiment, isocyanate is used as a solvent. Such methods are described, for example, in WO96/16028A1 (publ. 30.05.1996, BAYER AG [DE]), WO02/102763A1 (publ. 27.12.2002 BASF AG [DE]).

Phosgenation is carried out in any equipment known in the prior art. Examples of such equipment are, but are not limited to, statical mixers, described, for example, in U.S. Pat. No. 5,117,048A (publ. 26.05.1992, BAYER AG [DE]), U.S. Pat. No. 6,576,788B1 (publ. 10.06.2003, BASF AG [DE]); dynamic mixers described, for example, in EP2486975B1 (published 23 Sep. 2015, WANHUA CHEMICAL GROUP CO [CN]), U.S. Ser. No. 10/112,892B2 (published 30 Oct. 2018, COVESTRO DEUTSCHLAND AG [DE]); reactors having two or more zones, described, for example, in U.S. Pat. No. 7,851,648B2 (publ. 14.12.2010, BASF AG [DE]), RU2446151C2 (publ. 27.03.2012, BAYER MATERIAL SCIENCE AG [DE]), WO2012049158A1 (publ. 19.04.2012, BASF SE [DE]).

(Poly)diphenylmethane diisocyanate obtained using (poly)diaminodiphenylmethane according to the invention has a viscosity of from 150 to 250 mPa·s at 25° C.

### EXAMPLES OF EMBODIMENT OF THE INVENTION

### Example 1. Preparing a Dimer for Recycling

A commercially available sample of (poly)diaminodiphenylmethane was subjected to vacuum distillation to distill off dimeric components. The composition of the mixed dimers is shown in Table 2.

TABLE 2
Weight concentration of
components in wt. % *

| Compound | Content |
|---|---|
| 2,2′-DADPM | 0.15 |
| 2,4′-DADPM | 9.9 |
| 4,4′-DADPM | 84.4 |
| Impurities | the rest |

* according to GC-FID data

### Example 2. Dosing of Dimeric Components for Mixing with Aniline. Formaldehyde/Aniline (F/A) Molar Ratio of 0.465. Rearrangement at 95° C. (Variant 1 of the Method According to the Present Invention)

In a three-necked flask equipped with a mechanical stirrer, dropping funnel, thermocouple, and reflux condenser, an inert atmosphere is generated by blowing nitrogen (throughout the entire process), and aniline (200 g) is loaded into the flask. Then, 53.5 g of dimeric DADPM isomers (composition according to Table 2) are added to aniline and stirred until the dimer is completely dissolved. To the resulting mixture, 37.8% hydrochloric acid (77.82 g, 0.807 mol) is poured dropwise, in the course of which the reaction mixture is heated up to T_{r.m.}=50° C. and becomes yellow. The reaction mass is heated to 60° C., then 34.4% formalin (87.3 g, 1.076 mol) is added over 1 hour, preventing the reaction mass from heating up above 70° C. The mass is intensively (500 rpm) stirred with an overhead mechanical stirrer.

The temperature of the reaction mixture is brought to 70° C. and the mixture is kept at this temperature for 1 hour. Then the reaction mass is heated to 95-98° C. and stirred for 20 hours. After that, 50 ml of aniline are added for better separation of the condensation products. Then, the reaction mass is neutralized with sodium hydroxide. Alkali is taken with an excess of 5 wt. % relative to hydrochloric acid. After that, the reaction mass is transferred into a separatory funnel at 90° C., and the brine phase is decanted. The reaction mass is washed three times with 200 ml portions of hot water (60-80° C.).

Then an azeotropic mixture of water and aniline (1^{st}” fraction) and aniline (2^{nd} fraction) are distilled off from the product in a system for vacuum distillation. Then about 53.5 g of the fraction of dimeric diaminodiphenylmethane isomers are distilled off. The yield of residual polyamine is 188.5 g.

Synthesis conditions and properties of bottom products for the examples are given in Table 4.

### Example 3. Dosing of Dimeric Components after the Step of Formaldehyde Addition and Rearrangement at 95° C. F/A is 0.5 (Variant 2 of the Method According to the Present Invention)

In a four-necked flask equipped with a mechanical stirrer, thermocouple, reflux condenser, dual-flow mixer, and circulation loop with a direct cooling device, an inert atmosphere is created by blowing nitrogen (throughout the entire process) and aniline (200 g) is loaded into the flask. 35.6% hydrochloric acid (82.72 g) is added dropwise to the aniline by means of a peristaltic pump, in the course of which the reaction mass is heated up to T_{r.m.}=50° C. and becomes yellow. The reaction mass is cooled to 43° C. by circulation under the action of the peristaltic pump through a flexible circuit with a water-circulating cooling device, then 36.9% formalin (87.509 g) is added for 1 hour by means of a second peristaltic pump, preventing the reaction mass from warming up above 43° C. Simultaneously, a stream of cooled circulating reaction mixture is fed to the central nozzle of the mixer through the circulation loop. The circulating reaction mixture is withdrawn from the bottom of the flask, cooled in the cooling device, and returned to the flask from the top through the mixer. Both counter flows are partially mixed in the mixer. In addition, intensive mixing is carried out by an overhead mechanical stirrer. Immediately after termination of the dosing of formaldehyde, a mixture of DADPM dimers (53.76 g) is added in the form of a melt with a temperature of 100° C., while maintaining the temperature of 43° C.

The temperature of the reaction mass is brought to 53° C. and kept at this temperature for 1 hour. The reaction mass is heated to 95-98° C. and stirred for 20 hours. Then 25 ml of aniline are added for better separation of the condensation products. After that, the reaction mass is neutralized with sodium hydroxide. The alkali is taken with an excess of 5 wt % relative to hydrochloric acid for complete neutralization of hydrochloric acid and hydrolysis of formamides. After that, the reaction mass is transferred into a separatory funnel at 90° C. and the brine phase is decanted. The reaction mass is washed three times with 200 ml portions of hot water (60-80° C.).

Then an azeotropic mixture of water and aniline (1^{st} fraction) and aniline (2^{nd} fraction) are distilled off from the product in a vacuum distillation system. Distillation is carried out in a vacuum diaphragm pump in an oil bath. At the second step, a “trap to trap distillation” apparatus is assembled. The apparatus consists of a heated magnetic stirrer and a thermocouple, a bath with Wood's alloy, a 500 ml round-bottomed flask, a Würz nozzle, and a 500 ml round-bottomed two-necked receiver flask. The vapor temperature is monitored using a thermocouple. The vacuum is created using an oil vacuum pump. The residual aniline, other volatile components and about 55 g of a fraction of dimeric diaminodiphenylmethane (DADPM) isomers are distilled off. The composition of the combined fraction of dimers of Examples 2 and 3 is shown in Table 3:

TABLE 3
Weight concentration of components, %

| Compound | Content |
|---|---|
| Aniline | 0.16 |
| 2,2′-DADPM | 0.15 |
| 2,4′-DADPM | 5.97 |
| 4,4′-DADPM | 92.17 |
| Impurities | the rest |

A bottom polyamine is obtained, the product properties are shown in Table 4.

### Example 4. Adding Dimers after Dosing 100% of Formaldehyde and Rearranging Under Pressure (Variant 2)

In a four-necked flask equipped with a magnetic stirrer, a thermocouple, a reflux condenser, a nozzle for circulating a reaction mass and a formalin supply system, an inert atmosphere is generated by blowing nitrogen (throughout the entire process) and 200 g (2.151 mol) of pure aniline are loaded into the flask. To the resulting mixture, 61.260 g of 35.6% hydrochloric acid (0.634 mol) are added dropwise by means of a peristaltic pump, in the course of which the reaction mass is heated up to T_{r.m.}=50° C. and becomes yellow.

The temperature of the reaction mass is controlled so that it is about 43° C., then 91.008 g (1.118 mol) of 36.9% formalin is carefully added for 2.5 hours by means of a peristaltic pump, while preventing the reaction mass from warming up above 43° C. Simultaneously, a circulating reaction mixture stream is fed by means of a second peristaltic pump. Both counter flows are partially mixed in the nozzle. Then 38.057 g of DADPM dimer (composition according to Table 3) are added as a melt immediately after termination of the dosing of formaldehyde at 43° C. The temperature of the reaction mixture is raised to 80° C. stepwise (with the step of 5° C.) within 1 hour.

Then the reaction mass is loaded into a BUCHI reactor under a nitrogen atmosphere, where the reaction mixture is heated to 120° C. and kept for 2 hours after reaching said temperature.

Then, 61.260 g (0.643 mol) of 42.0% NaOH solution is loaded into the reactor and neutralization is performed at 120° C. for 30 minutes, then the lower inorganic layer is drained. After that, the reaction mass is washed three times with 200 ml portions of hot distilled water at 90° C.

Aniline and DADPM isomers are then distilled off similarly to examples 1 and 2. The properties of a bottom product are shown in Table 4.

### Example 5. Rearrangement at 130° C. (Variant 2)

The synthesis is carried out similarly to example 4, but the final keeping of the reaction mixture is carried out at 130° C.

### Example 6. Addition of Dimers after Dosing of 100% of Formaldehyde and Rearrangement Under Pressure. F/A is 0.526 (Variant 2)

In a four-necked flask equipped with a magnetic stirrer, a thermocouple, a reflux condenser, a nozzle for circulating a reaction mass and a formalin supply system, an inert atmosphere is generated by blowing nitrogen (throughout the entire process), and 400 g (4.295 mol) of pure aniline are loaded into the flask. To the resulting mixture, 129.763 g (1.267 mol) of 35.6% hydrochloric acid are added dropwise by means of a peristaltic pump, in the course of which the reaction mass is heated up to T_{r.m.}=50° C. and becomes yellow.

The temperature of the reaction mass is controlled such that it is about 43° C., then 184.116 g (2.262 mol) of 36.9% aqueous formalin are carefully added for 3 hours by means of a peristaltic pump, while preventing the reaction mass from warming up above 43° C. Simultaneously, a circulating reaction mixture stream is fed by means of a second peristaltic pump. Both counter flows are partially mixed in the nozzle. Then 76.178 g of DADPM dimer (composition according to Table 3) are added as a melt immediately after termination of the dosing of formaldehyde at 43° C. The temperature of the reaction mixture is raised to 80° C. stepwise (with the step of 5° C.) within 1 hour.

Then, the reaction mass is loaded into a BUCHI reactor in a nitrogen atmosphere, where the reaction mixture is heated to 120° C. and kept for 1 hour after reaching said temperature.

After that, 131.659 g (1.356 mol) of 41.2% alkali solution is loaded into the reactor, and neutralization is performed at 120° C. for 30 minutes, then the lower inorganic layer is drained. The reaction mass is then washed three times with 200 ml portions of hot distilled water.

Then, aniline and DADPM are distilled off similarly to examples 1 and 2. The properties of a bottom product are shown in Table 4.

### Example 7. Addition of Dimers after Dosing of 80% of Formaldehyde and Rearrangement Under Pressure (Variant 2)

In a four-necked flask equipped with a magnetic stirrer, a thermocouple, a reflux condenser, a formalin mixing unit and a reaction mass circulation loop, an by means inert atmosphere is generated by blowing nitrogen (throughout the entire process), and 400 g (4.295 mol) of aniline are loaded into the flask. To the resulting mixture, 129.763 g (1.267 mol) of 35.6% hydrochloric acid are added dropwise by means of a peristaltic pump, in the course of which the reaction mass is heated up to T_{r.m.}=50° C. and becomes yellow.

By circulation through the cooling loop, the temperature of the reaction mixture is maintained so that it is about 43° C., then 147.293 g (1.810 mol) of 36.9% formalin are carefully dosed (for 3 hours) by means of a peristaltic pump, preventing the reaction mass from warming up above 43° C. Simultaneously, a circulating reaction mixture stream is fed by means of a second peristaltic pump. Both counter flows are partially mixed in the nozzle. Then 76.178 g of DADPM dimer (composition according to Table 3) are added in the form of a melt immediately after termination of the dosing of the first portion of formaldehyde, at 43° C. The temperature is raised to 53° C., the reaction mass is kept at this temperature for 30 minutes and then the remaining formalin (36.823 g, 0.452 mol) is added.

The temperature of the reaction mass is raised to 80° C. stepwise (with the step of 5° C.) within 1 hour, while recording the heating time.

After that, the reaction mass is loaded into a BUCHI reactor in a nitrogen atmosphere, where the reaction mixture is heated to 120° C. and kept for 1 hour.

Then, 131.659 g (1.356 mol) of 41.2% alkali solution are loaded into the reactor, and neutralization is performed at 120° C. for 30 minutes, then the lower inorganic layer is drained. The reaction mass is then washed three times with 200 ml portions of hot distilled water at 90° C.

Then, aniline and DADPM isomers are distilled off similarly to examples 2 and 3. The properties of a bottom product are shown in Table 4.

### Example 8. Fractional Addition of Dimers and Formaldehyde, and Rearrangement Under Pressure (Variant 2)

A 60 L glass-lined De Dietrich reactor with a heat-exchange jacket and a stirrer is charged with 0.762 kg of an DADPM solution in 20.00 kg of aniline with the content of DADPM components as shown in Table 5. The reactor is charged with 6.39 kg of 36.1% hydrochloric acid, and the mixture is cooled to 35° C. 8.122 kg of a 35.5% formalin are dosed, while maintaining the temperature at 35° C. Thereafter, 3.046 kg of an DADPM melt are dosed into the reactor. Then the reaction mass is heated to 51° C. and kept for 1 hour at this temperature. After that, 1.433 kg of a 35.5% formalin are dosed, while maintaining the temperature at 51° C. Then the mixture is heated to 140° C. and kept for 15 minutes at this temperature. Then, 3.0 kg of aniline are added to the mixture for dilution. After that, 7.14 kg of a 42% NaOH solution are added to the reactor and stirred at 110° C. for 2 hours. The mixture is cooled to 90° C., settled for 45 minutes, and the lower phase is separated. The pDADPM phase is washed twice with 20 kg H₂O each time. The washed pDADPM is subjected to stepwise distillation in a series of columns for the distillation of water, aniline, and dimeric DADPM. A polyamine bottom has a viscosity of 103 mPa·s at 90° C.

TABLE 4
Results of the analyzes of products according to the example

| Parameter | | Example 2 | Example 3 | Example 4 | Example 5 | Example 6 | Example 7 | Example 8 |
|---|---|---|---|---|---|---|---|---|
| MDA weight/aniline weight | | 0.2675 | 0.2688 | 0.1903 | 0.1903 | 0.1904 | 0.1904 | 0.1904 |
| Formaldehyde/aniline Molar ratio | | 0.465 | 0.5 | 0.520 | 0.521 | 0.527 | 0.527 | 0.526 |
| Cl/N molar ration | | 0.3 | 0.3 | 0.25 | 0.25 | 0.25 | 0.25 | 0.25 |
| Weight fractions of | 4,4-MDA | 47.79 | 54.78 | 50.24 | 49.73 | 52.98 | 44.79 | 44.73 |
| main products, % wt. | 2,4-MDA | 3.38 | 3.03 | 2.81 | 3.19 | 2.40 | 2.20 | 2.28 |
| norm. per 100%* | M3A | 27.14 | 25.84 | 26.65 | 27.39 | 24.49 | 27.59 | 27.58 |
| | M4A | 12.29 | 9.81 | 11.54 | 11.64 | 10.55 | 13.07 | 12.26 |
| | M5A | 5.94 | 4.65 | 5.43 | 5.50 | 5.26 | 6.86 | 6.35 |
| | M6A | 2.40 | 1.26 | 2.31 | 1.95 | 2.94 | 3.49 | 5.31 |
| | M7A | 0.80 | 0.48 | 0.84 | 0.60 | 1.04 | 1.46 | 1.03 |
| | M8A | 0.26 | 0.16 | 0.18 | 0.00 | 0.34 | 0.54 | 0.46 |
| Tetramer/trimmer | M4A/M3A | 0.453 | 0.380 | 0.433 | 0.425 | 0.431 | 0.474 | 0.444 |
| weight ratio | | | | | | | | |
| Weight ratio of trimer | M3A/M2A | 0.530 | 0.447 | 0.502 | 0.517 | 0.442 | 0.587 | 0.587 |
| to the sum of dimers | | | | | | | | |

| | | | | | | | |
|---|---|---|---|---|---|---|---|
| Viscosity at 90° C., mPa · s | 84.6 | 59.5 | 77.3 | 90.7 | 86.4 | 115 | 103 |
| Aniline content, ppm | 10 | 20 | 10 | 10 | 10 | 10 | 10 |
| Weight fraction of NH groups of | 0.05 | 0.06 | 0.04 | 0.04 | 0.05 | 0.03 | 0.03 |
| secondary amines,** % | | | | | | | |

*According to HPLC data;
**According to NMR data.

When comparing the results of different examples, it can be seen that despite lower formaldehyde-aniline ratio in Example 2 (0.465) than in Example 3 (0.5), the amine product in Example 3 is lighter and has a low tetramer-trimer ratio (0.38 versus 0.45). The inventors believe that this is due to the method of introducing the recycled dimer in Example 3 after dosing of formaldehyde, which reduces the likelihood of the formation of heavy oligomers in the starting steps of the reaction.

In Examples 4 and 5, the formaldehyde-aniline ratio was increased to 0.52, while the weight ratio of recycled DADPM to feed aniline was reduced to 0.19, compared to 0.27 in Examples 2 and 3.

In this case, the tetramer-trimer ratio slightly increases to 0.43, with the trimer-dimer ratio increased to 0.50-0.52.

The highest formaldehyde-aniline ratio was used in Examples 6-8. In this case, the tetramer-trimer ratio relatively strongly (up to 0.474) increased only in Example 7, where 80% of formaldehyde was dosed before adding recycled dimers, and 20% of formaldehyde was added after dosing recycled dimers and a short-time keeping. In this case, the polyamine of Example 7 is most viscous of the series (115 mPa·s at 90° C.) with a dimer content of 47%. In example 8, when 20% of the dimers was dosed in step 1, the M4/M3 ratio was 0.44 and the viscosity was 103 mPa·s at 90° C.

### Example 9. Phosgenation

Phosgene (68 g, 0.68 mol), in the form of a 20% solution in toluene (340 g of solution), is poured into a 2000 ml three-necked round-bottomed flask, cooled with a mixture of ice and salt under slight nitrogen breathing. A solution of polyamine obtained according to the corresponding example (15.5 g, about 0.02 mol of amino groups) in 100 g of chlorobenzene is added dropwise to the stirred solution at −2° C., over 30 minutes. The rate of the addition is adjusted such that the temperature of the reaction mixture does not exceed 0-5° C., for 15 minutes. Then the dropping funnel is washed with another 100 g of chlorobenzene. The resulting suspension is slowly stepwise heated to 98° C. within 2.5-3 hours to separate hydrogen chloride. Waste gases (hydrogen chloride, phosgene) are captured in a trap system with a 10% NaOH solution, while solvent vapors are condensed in a reflux condenser. After dissolution of the precipitate, the system is purged with a stream of nitrogen directed to the mass of the solution at 122° C. for 30 min, with a reflux condenser. The solution is evaporated under vacuum to a volume of 20 ml, and the residue is heated in an oil bath under a vacuum of 0.23 mbar at 180° C. for 30 min.

The product is analyzed. The results of analysis of the products are shown in Table 5.

TABLE 5
Results of analyzes of pMDI products

| Polyamine source | Example 2 | Example 6 | Example 7 |
|---|---|---|---|
| Isocyanate, viscosity, | 163.5 | 132.6 | 182.3 |
| mPa · s at 25° C. | | | |
| Isocyanate, hydr. | 0.17 | 0.14 | 0.10 |
| chlorine, % | | | |
| Isocyanate, NCO, % | 32.5 | 33.2 | 30.9 |
| Phenyl isocyanate, ppm | 29 | 20 | 14 |

### Example 10. Comparative

Polyamine is synthesized according to U.S. Pat. No. 4,792,624 with recycling the final polyamine. Polyamine products are obtained with a tetramer/trimer ratio of 0.52 and a trimer/dimer ratio of 0.46.

From the presented examples, it follows that the recycle of dimers of diaminodiphenylmethanes according to the present invention, instead of the recycle of the final polyamine according to the prior art (U.S. Pat. No. 4,792,624), can significantly reduce the tetramer/trimer weight ratio from 0.52 to about 0.38-0.48, and keeping the trimer/dimer weight ratio above 0.45, i.e. significantly increase the proportion of the most valuable trimeric oligomers in the final polyamine. In turn, the use of such polyamines enriched with trimeric oligomers for the synthesis of polyisocyanates by phosgenation provides polyisocyanates with a controlled viscosity and a reduced content of phenyl isocyanates, as illustrated in example 9.

1. (Poly)diaminodiphenylmethane having an oligomeric distribution characterized by a tetramer/trimer weight ratio of not more than 0.48 and a trimer/dimer weight ratio of not less than 0.45, and having a viscosity of 50-150 mPa·s at 90° C.

2. (Poly)diaminodiphenylmethane according to claim 1 having an oligomeric distribution characterized by a tetramer/trimer weight ratio of not more than 0.42.

3. (Poly)diaminodiphenylmethane according to claim 2 having an oligomeric distribution characterized by a trimer/dimer weight ratio of not less than 0.53.

4. A method for producing (poly)diaminodiphenylmethane, comprising the following steps:
1) mixing HCl and aniline and a mixture of recycled dimeric diaminodiphenylmethane (DADPM) isomers;
2) reacting the mixture of said aniline, dimeric diaminodiphenylmethane DADPM isomers and HCl with an aqueous solution of formaldehyde at a temperature of not higher than 70° C.;
3) raising the temperature of the reaction mass obtained in step (2) to a temperature below or equal to 140° C. and keeping the mass at this temperature;
4) neutralizing acidic compounds in the mass obtained in step (3);
5) washing the mass obtained in step (4) with water;
6) distilling off low-boiling components and aniline from the mass obtained in step (5);
7) distilling off the mixture of dimeric diaminodiphenylmethane (DADPM) isomers from the mass obtained in step (6),
characterized in that a stream of the mixture of dimeric diaminodiphenylmethane (DADPM) isomers obtained in step (7) is recycled to step (1).

5. The method according to claim 4, wherein the mixture of dimeric diaminodiphenylmethane (DADPM) isomers is a mixture of dimeric diaminodiphenylmethanes with a content of 4,4′-DADPM of not less than 50%.

6. The method according to claim 5, wherein HCl is hydrochloric acid or hydrogen chloride gas, preferably hydrochloric acid, more preferably 31-38% hydrochloric acid.

7. The method according to claim 4, wherein the preferred temperature in step (2) ranges from 20° C. to 70° C.

8. The method according to claim 4, wherein in step (3) the temperature is raised to values in the range of 80-140° C.

9. A method for producing (poly)diaminodiphenylmethane, comprising the following steps:
1) mixing HCl and aniline with addition of a part of a recycled mixture of dimeric diaminodiphenylmethane (DADPM) isomers of from 0 to 25 wt. % based on the total amount of the added recycled mixture;
2) reacting the mixture from step (1) with a first part of an aqueous formaldehyde solution at a temperature T₁ of not higher than 70° C., wherein said first part of an aqueous formaldehyde solution is from 50 to 100% of the total amount of formaldehyde added to the reaction;
3) adding the remaining part of the recycled mixture of dimeric diaminodiphenylmethane isomers up to reaching the total amount of DADPM isomers of not more than 25 wt % of the initial weight of aniline;
4*) in the case when in step (2) the introduced amount of formaldehyde is less than 100% of the total amount of formaldehyde introduced into the reaction, adding a second part of formaldehyde of from more than 0 to up to 50% of the total amount of formaldehyde introduced into the reaction;
4) keeping the reaction mass obtained in step (3) or, in the case when step (4*) is carried out, the reaction mass obtained in step (4*) at a temperature T2 of not higher than 80° C.;
5) raising the temperature of the reaction mass obtained in step (4) to a temperature below or equal to 140° C. and keeping the mass at this temperature;
6) neutralizing acidic compounds in the mass obtained in step (5);
7) washing the mass obtained in step (6) with water;
8) distilling off low-boiling components and aniline from the mass obtained in step (7); and
9) distilling off the mixture of dimeric diaminodiphenylmethane isomers from the mass obtained in step (8),
characterized in that the mixture of dimeric diaminodiphenylmethane isomers obtained in step 9 is recycled to step (1) and/or step (3).

10. The method according to claim 9, wherein HCl is hydrochloric acid or hydrogen chloride gas, preferably hydrochloric acid, more preferably 31-38% hydrochloric acid.

11. The method according to claim 9, wherein in step (3) the addition of the remaining part of the recycled mixture of isomeric diaminodiphenylmethanes (DADPM) is carried out until the total amount of DADPM isomers reaches the value of from 5% to 25% by weight of the initial weight of aniline.

12. The method according to claim 9, wherein step (4*) is carried out at a temperature of from 30° C. to 80° C.

13. The method according to claim 9, wherein the keeping in step (4) is carried out at a temperature of from 30° C. to 80° C.

14. The method according claim 9, wherein in step (5) the temperature is raised to the range of 80-140° C.

15. The method according to claim 9, wherein the mixture of dimeric diaminodiphenylmethane isomers is a mixture of dimeric diaminodiphenylmethanes with a content of 4,4′-DADPM of not less than 50%.

16. The method according to claim 4, wherein the mixing in step (1) is carried out at a molar ratio of chlorine contained in HCl to the total nitrogen contained in aniline and recycled DADPM isomers (Cl/N) of (0.2÷0.4)/1.

17. A (poly)diaminodiphenylmethane obtained by the method according to claim 4 and having an oligomeric distribution characterized by a tetramer/trimer weight ratio of not more than 0.48 and a trimer/dimer weight ratio of not less than 0.45, and having a viscosity of 50-150 mPa·s at 90° C.

18. A method for producing (poly)diphenylmethane diisocyanate, comprising phosgenating the (poly)diaminodiphenylmethane according to claim 1.

19. (Poly)diphenylmethane diisocyanate obtained by the method according to claim 18.

20. The (poly)diphenylmethane diisocyanate according to claim 19, characterized by a viscosity of 150-250 mPa·s at 25° C.
